(19) United States
(12) Patent Application Publication
(10) Pub. No.: US 2005/0163328 A1
(43) Pub. Date: Jul. 28, 2005

(54) Fitting of parameters in an electronic device

(76) Inventor: Soren Colding, Hellerup (DK)

Correspondence Address: Dykema Gossett, Suite 300 West, 1300 I Street NW, Washington, DC, 20005-3306, US

(21) Appl. No.: 10/508,214

(22) PCT Filed: Mar. 19, 2003
(86) PCT No.: PCT/DK03/00187

(30) Foreign Application Priority Data
Mar. 20, 2002 (DK) PA 2002 00432

(51) Int. Cl. H04R 29/00; A61B 5/00

(52) U.S. Cl. 381/60; 600/559

(57) ABSTRACT

The invention relates to a method for fitting an electronic device too the needs of a user, where a fitting device is provided, which includes a display and means for pointing a specific area in the display, where in the display a block including a set of trimmers for manipulating one or more parameters in the electronic device is available and where in the display a number of fields are indicated for manipulation of parameters in the electronic device, where the trimmer block upon pointing on a specific field is linked to this field for manipulating the parameter related to this field.

### AREA OF THE INVENTION

The invention relates to methods and systems for fitting audio devices, e.g. hearing aids or headsets, to a specific need or desire of a user of such. More particularly the invention relates to an electronic device including a screen, e.g. a conventional computer, which when operatively connected to the audio device can be used to manipulate the settings of the audio device and hence modify the transfer characteristics.

### BACKGROUND OF THE INVENTION

Such electronic devices are well known and are used in connection with most programmable hearing devices.

The complexity of the electronic devices is significant and the complexity is increasing. Therefore there is a need for improvement of such devices in order to achieve devices, which are simpler to handle.

The objective of the present invention is therefore to provide a method and a device for use in fitting audio devices, which device while maintaining the required technical capabilities is simpler to handle.

### SUMMARY OF THE INVENTION

According to the invention the objective of the present invention is achieved by means of the method as defined in claim 1.

By this method it will always be obvious to the user which function is presently being manipulated in the audio device. At the same time the trimmer button may be designed larger and hence enable easier use and still occupy limited space. The significant reduction in trimmer buttons at the same time makes the display simpler and easier to use. The improvement inherently results in a more reliable fitting process and hence increased user satisfaction.

Advantageous embodiments are indicated in claims 2-4.

According to the invention the objective of the invention is likewise achieved by means of the program as defined in claim 5 and being capable of performing the method as mentioned above.

Advantageous embodiments are indicated in claims 6-8.

In connection with a computer the same advantages as mentioned in connection with the method are hereby achieved.

### BRIEF DESCRIPTION OF THE DRAWINGS

FIG. 1 is a schematic drawing showing the graphic user interface in a first position of the trimmers where the adjustment of a first area is allocated;

FIG. 2 is a schematic drawing showing the graphic user interface in a second position of the trimmers where the adjustment of a second area is allocated.

### DESCRIPTION OF A PREFERRED EMBODIMENT

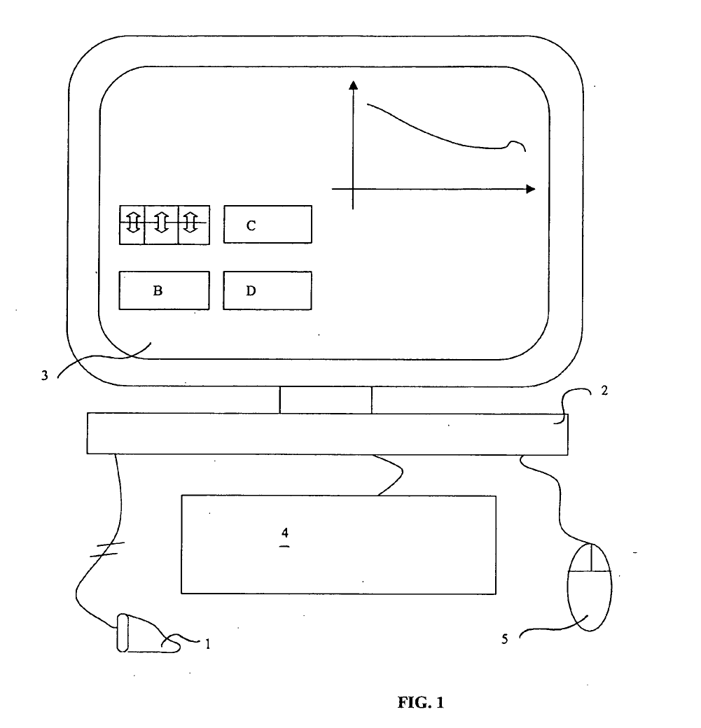

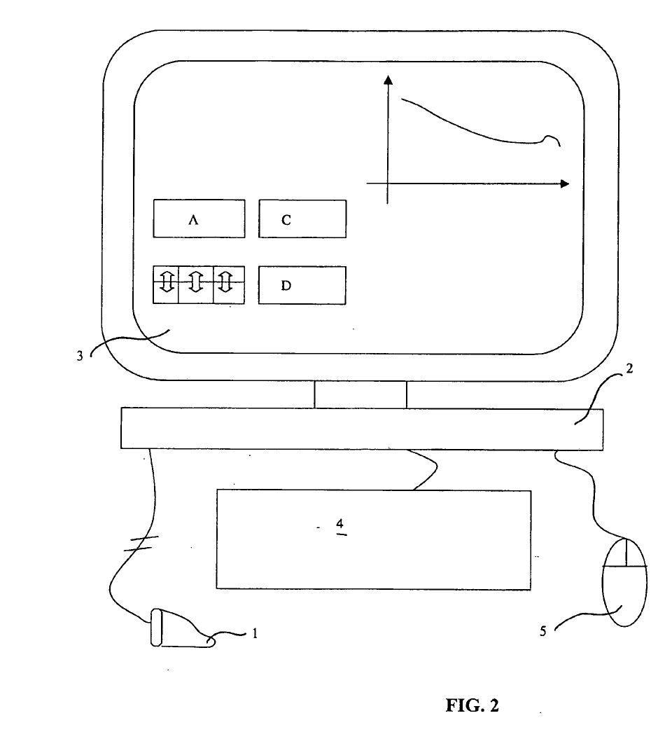

From FIGS. 1 and 2 a hearing aid appears in connection with fitting equipment adapted to adjust the parameters of the hearing aid. The fitting equipment comprises a computer with a screen, a keyboard and a mouse. The computer runs a fitting software program that is capable of generating control parameters to the hearing aid in order to change settings of this and hereby change the transfer characteristics of the hearing aid.

The software generates an output to the screen. In the FIGS. 1 and 2 four fields A,B,C,D are indicated for manipulating parameters in the hearing aid, the fields indicating areas of low frequencies and soft gain, low frequencies and loud gain, high frequencies and soft gain, high frequencies and loud gain, respectively. A block of trimmers is likewise indicated. The block includes three sets of trimmers, where one of these sets is adapted for manipulating left ear hearing aid, one is adapted for manipulating the right ear hearing aid and the last set of trimmers, the one in the middle, is adapted for manipulating the parameters in both the left and the right ear hearing aid with an identical increase or decrease of the same parameter. This is of course only relevant when two hearing aids are fitted simultaneously.

In FIG. 1 the field A has been activated and the trimmer block is indicated in the position of a first selected field A, hereby indicating visually that this field, or rather the parameter connected to this field, is active. In FIG. 2 yet another field B has been activated and the trimmer block has been moved correspondingly. This gives the person working with the adjustment a very clear and simple indication of what is being done and hence facilitates the process and may reduce the occurrence of errors in the process.

The trimmer block may be allocated to adjust parameters related to more than one field, e.g. by dragging the trimmer block to an area in-between two fields or by pointing between two fields and hence effect the movement of the trimmer block to this area. The trimmer block may then overlap at least part of two or more fields and hereby clearly indicate that the trimmers are active for the fields partly overlapped. This further facilitates the fitting process.

1. A method for fitting an electronic device to the needs of a user, wherein a fitting device is provided for performing the method, wherein the fitting device includes a display and means for pointing a specific area in the display, wherein in the display a block including a set of trimmers for manipulating one or more parameters in the electronic device is displayed and wherein further in the display a number of fields are indicated each relating to a specific parameter in the electronic device, wherein the trimmer block is linked to at least one field for manipulating the parameter related to this field and wherein the trimmer block allocation to one or more fields may be altered using the pointing means.

2. A method according to claim 1, wherein the trimmer block in the display is moved to a specific field by dragging this using the pointing means.

3. A method according to claim 1, wherein the trimmer block in the display is moved to a specific field by pointing at this using the pointing means, whereby the trimmer block will appear in this field.

4. A method according to claim 1, wherein the trimmer block in the display upon pointing between two or more fields is displayed over an area of these fields and is linked to the parameters of these fields.

5. A program for use in a fitting device which includes a display and means for manipulating/controlling a specific area in the display, the program being stored on a computer readable media, the program comprising program code for generating a display having two or more fields indicating a specific parameter to be adjusted, program code for generating a set of trimmers capable of increasing and/or decreasing a parameter to be adjusted, program code for enabling selection of a specific field, and program code for linking the set of trimmer to a parameter in a selected field.

6. A program according to claim 4, wherein program code is included for displaying the set of trimmer at the location of the field selected.

7. A program according to claim 5, comprising program code for moving the trimmer block in the display to a specific field by pointing at this using the pointing means, whereby the trimmer block will appear in this field.

8. A program according to claim 5, comprising program code which upon pointing between two or more fields will effect display of the trimmer block over an area of these fields and program code that links the trimmer block to the parameters of these fields.

9. A program according to claim 5, wherein the program is stored on a storage medium.
